(19) United States
(12) Patent Application Publication
(10) Pub. No.: US 2020/0270638 A1
(43) Pub. Date: Aug. 27, 2020

(54) EXOSOMES AS A VECTOR FOR GENE DELIVERY IN RESISTANCE TO NEUTRALIZING ANTIBODY AND METHODS OF THEIR MANUFACTURE

(71) Applicant: ICAHN SCHOOL OF MEDICINE AT MOUNT SINAI, New York, NY (US)

(72) Inventors: Susmita SAHOO, New York, NY (US); Roger J. HAJJAR, New York, NY (US); Yaxuan LIANG, New York, NY (US)

(73) Assignee: ICAHN SCHOOL OF MEDICINE AT MOUNT SINAI, New York, NY (US)

(21) Appl. No.: 16/762,569

(22) PCT Filed: Nov. 8, 2018
(86) PCT No.: PCT/US2018/059813

§ 371 Date: May 8, 2020

Related U.S. Application Data
(60) Provisional application No. 62/583,117, filed on Nov. 8, 2017.

(51) Int. Cl. C12N 15/86

(52) U.S. Cl. CPC C12N 15/86; C12N 2750/14151; C12N 2750/14143

(57) ABSTRACT

Provided is a method of making isolated exosomes containing an adeno-associated viral (AAV) vector, including disposing a suspension on an iodixanol gradient, wherein the suspension includes exosomes containing AAV vectors and the iodixanol gradient includes a higher layer of between 20% and 30% w/v iodixanol solution, an intermediate layer of between 30% and 50% w/v iodixanol solution, and a lower layer of between 50% and 70% w/v iodixanol solution, and centrifuging the iodixanol gradient at between 200,000 g and 300,000 g for at least 2 hours.

### CROSS-REFERENCE TO RELATED APPLICATIONS

This application is an International Application filed under the Patent Cooperation Treaty, and claims priority to U.S. Provisional Patent Application No. 62/583,117, filed Nov. 8, 2017, the entire contents of which are incorporated herein by reference.

### FIELD OF THE INVENTION

The subject matter disclosed herein relates to compositions for delivering viral vectors and methods for their manufacture. More particularly, this disclosure relates to adeno-associated viral (AAV) vectors packaged in exosomes (AAVExo) and methods for making isolated preparations of AAVExo.

### BACKGROUND OF THE INVENTION

Exosomes are nano-sized extracellular vesicles secreted from almost all cell types. Cargos carried within exosomes include specific proteins and RNAs that are transferred to recipient cells in the vicinity or at a distance. Recent studies indicate exosomes as a natural carrier for virus including hepatitis A virus and hepatitis C virus. Interestingly, AAVs are also naturally secreted via exosomes. Naturally present neutralizing antibody (Nab) in part of the human population poses significant challenge to develop AAVs as ideal vecots for effective gene delivery for clinical usage. Exosomes can naturally envelope AAV-vectors to shield from Nab. Therefore, AAV-containing exosomes (AAVExo) can be superior agents for delivering genes to cells, tissues, and organs. The robust exosomal membrane can protect AAVs from access by neutralizing antibodies and therefore AAVExo can present higher resistance to Nab. However, currently available methods for making AAVExo results in a disadvantageously high level of contamination of AAVExo with AAV not contained in exosomes. Complications from such contamination include lower levels of transfection, perhaps due in part to increased Nab susceptibility owing to the presence of free AAV. Thus, an improved method for making and isolated AAVExo is needed.

The present disclosure is directed to overcoming these and other deficiencies in the art.

### SUMMARY OF THE INVENTION

In one aspect, disclosed is a method of making isolated exosomes containing an adeno-associated viral (AAV) vector, including disposing a suspension on an iodixanol gradient, wherein the suspension includes exosomes containing AAV vectors and the iodixanol gradient includes a higher layer of between 20% and 30% w/v iodixanol solution, an intermediate layer of between 30% and 50% w/v iodixanol solution, and a lower layer of between 50% and 70% w/v iodixanol solution, and centrifuging the iodixanol gradient at between 180,000 g and 300,000 g for at least 2 hours. In some embodiments, the method further includes, before disposing the suspension on the iodixanol gradient, forming a sucrose suspension by suspending exosomes containing AAV vectors in a 30% w/v solution of sucrose in deuterium oxide and centrifuging the first suspension at between 80,000 g and 120,000 g for at least 70 min to form a fraction of exosomes containing AAV vectors, washing the fraction, and suspending the fraction in a solution that does not contain sucrose.

In some examples, cells in which the exosomes containing AAV vectors were produced include 293T cells, Per.C6 cells, AGE1.CR cells, AGE1.HN cells, KG-1 cells, human umbilical vein endothelial cells (HUVECs), embryonic stem (ES) cells from humans, human mesenchymal stem cells (MSCs), human fibroblasts, human keratinocytes, human hematopoietic progenitor cells, human cKit+ stem cells, human cardiosphere-derived cells, HeLa cells, induced pluripotent cells, K562 cells, Caco-2 cells, human T-cells, human dendritic cells, human smooth muscle cells, or endothelial cells derived from vascular origin. In other examples, the lower layer includes 60% w/v iodixanol solution, the intermediate layer includes 40% w/v iodixanol solution, and the higher layer includes 25% w/v iodixanol solution. Further examples include centrifuging the iodixanol gradient at 250,000 g, or between 180,000 and 200,000 g, or centrifuging the iodixanol gradient for at least 3 hours.

Still further examples include collecting a final fraction from the iodixanol gradient after centrifuging the iodixanol gradient, wherein the final fraction includes a highest portion of the first layer and the portion is up to one half volume of the higher layer. In other examples the portion is up to one quarter volume of the higher layer. In yet other examples the portion is up to one eighth volume of the higher layer. In still other examples, more than 95% of AAV vector genome copies present in the final fraction are in exosomes. In other embodiments, the AAV vectors are AAV1, AAV2, AAV6, or AAV9 vectors.

In another aspect, disclosed is a method of transfecting a cell, comprising contacting the cell with exosomes containing an adeno-associated viral (AAV) vector, wherein a method for making the exosomes containing an AAV vector includes disposing a suspension on an iodixanol gradient, wherein the suspension includes exosomes containing AAV vectors and the iodixanol gradient includes a higher layer of between 20% and 30% w/v iodixanol solution, an intermediate layer of between 30% and 50% w/v iodixanol solution, and a lower layer of between 50% and 70% w/v iodixanol solution, and centrifuging the iodixanol gradient at between 180,000 g and 300,000 g for at least 2 hours. In some embodiments, the method further includes, before disposing the suspension on the iodixanol gradient, forming a sucrose suspension by suspending exosomes containing AAV vectors in a 30% w/v solution of sucrose in deuterium oxide and centrifuging the first suspension at between 80,000 g and 120,000 g for at least 70 min to form a fraction of exosomes containing AAV vectors, washing the fraction, and suspending the fraction in a solution that does not contain sucrose.

In some examples, cells in which the exosomes containing AAV vectors were produced include 293T cells, Per.C6 cells, AGE1.CR cells, AGE1.HN cells, KG-1 cells, human umbilical vein endothelial cells (HUVECs), embryonic stem (ES) cells from humans, human mesenchymal stem cells (MSCs), human fibroblasts, human keratinocytes, human hematopoietic progenitor cells, human cKit+ stem cells, human cardiosphere-derived cells, HeLa cells, induced pluripotent cells, K562 cells, Caco-2 cells, human T-cells, human dendritic cells, human smooth muscle cells, or endothelial cells derived from vascular origin. In other examples, the lower layer includes 60% w/v iodixanol solution, the intermediate layer includes 40% w/v iodixanol solution, and the higher layer includes 25% w/v iodixanol solution. Further examples include centrifuging the iodixanol gradient at 250,000 g, or between 180,000 g and 200,000 g, or centrifuging the iodixanol gradient for at least 3 hours.

Still further examples include collecting a final fraction from the iodixanol gradient after centrifuging the iodixanol gradient, wherein the final fraction includes a highest portion of the first layer and the portion is up to one half volume of the higher layer. In other examples the portion is up to one quarter volume of the higher layer. In yet other examples the portion is up to one eighth volume of the higher layer. In still other examples, more than 95% of AAV vector genome copies present in the final fraction are in exosomes. In other embodiments, the AAV vectors are AAV1, AAV2, AAV6, or AAV9 vectors.

In a particular aspect, disclosed is a method of making isolated exosomes containing an adeno-associated viral (AAV) vector, including forming a sucrose suspension by suspending exosomes containing AAV vectors in a 30% w/v solution of sucrose in deuterium oxide and centrifuging the first suspension at 100,000 g for at least 70 min to form a fraction of exosomes containing AAV vectors, washing the fraction, and suspending the fraction in a solution that does not contain sucrose, forming a suspension, disposing a suspension on an iodixanol gradient, wherein the suspension comprises exosomes containing AAV vectors and the iodixanol gradient comprises a higher layer of 25% w/v iodixanol solution, an intermediate layer of 40% w/v iodixanol solution, and a lower layer of 60% w/v iodixanol solution, centrifuging the iodixanol gradient at 250,000 g for at least 3 hours, and collecting a final fraction from the iodixanol gradient after centrifuging the iodixanol gradient, wherein the final fraction includes a highest portion of the first layer and the portion is up to one eighth volume of the higher layer, wherein the AAV vectors are AAV9 vectors and cells in which the exosomes containing AAV vectors were produced comprise 293T cells.

In yet another aspect, disclosed is a suspension including exosomes containing adeno-associated viral (AAV) vectors, wherein 95% of more of AAV vector genome copies present in the suspension are in exosomes. In one embodiment, the AAV vectors are AAV1, AAV2, AAV6, or AAV9 vectors. In a particular example, the AAV vectors are AAV9 vectors.

### BRIEF DESCRIPTION OF THE DRAWINGS

These and other features, aspects, and advantages of the present disclosure will become better understood when the following detailed description is read with reference to the accompanying drawings, wherein:

FIGS. 1A-1C show A) a nonlimiting example of a method flow chart of AAVExo Purification. B) Conditioned medium of AAV-producing 293T cells was analyzed to measure particle size by DLS. Population of exosomes with ˜100 nm in size was detected as indicated by arrow. C) Purified AAVExo was analyzed by DLS.

FIGS. 2A-2D show strategies and characterization of AAVExo purification. Equal titer of pure AAV (A, control-1), a mixture of empty exosome & free AAV (B, control-2), and AAVExo (C) crude prep were loaded on top of an Optiprep density gradient and ultracentrifuged at 250,000×g for 3 h. Separated fractions were collected from the top as indicated and analyzed for the presence of exosomes (1. size, by DLS; 2.for exosomes marker protein, flotillin-1 by WB) and for the presence of AAV (by qPCR). D) Optiprep density gradient after ultracentrifugation. A sharp white band was found in ˜25% layer both in AAVExo and empty Exo & pure AAV control tubes. The white band was collected in F4.

FIGS. 3A-3D show Fraction 4 has the major amount of clean AAVExo compared to fraction 3. A) Exosome size distribution in F3 and F4 of AAVExo measured by NTA. B) Exosome concentration of F3 and F4 in control-2 and AAVExo measured by NTA. C) F4 but not F3 has effective transduction capacity in vitro. 293T cells were treated with same titer of AAV9Exo F3 and F4 for 5 days. EGFP was detected by fluorescence microscopy. D) AAV-containing exosomes in multivesicular body. AAV-producing cells were imaged by TEM, and AAV particles were observed in exosomes prior to secretion. Lower panel represents the zoomin area of upper panel. Scale bar, 200 nm.

FIGS. 4A-4E show AAVExo has stronger capacity of gene delivery compared to free AAVs in vitro and in vivo. A) 293T cells were treated with same titer of AAV9Exo-EGFP or pure AAV9-EGFP as a control for 5 days. Cells were imaged with fluorescence microscopy. B) The same cells after fluorescence microscopy were then analyzed by flow cytometry. C) Experiment flowchart showing that wild type mice were intramyocardial injected with PKH67 labeled AAVExo or negative dye control. 1.5 h after injection, mice hearts were harvested and digested. Cardiomyocytes and non-cardiomyocytes were isolated and analyzed by flow cytometry. D) AAVExo is taken up rapidly by cardiac cells in vivo detected by flow cytometry. E) Same titer)(4.41E¹⁰ of AAVExo-mCherry or AAV-mCherry were intramyocardial injected to wild type mice. After two weeks, mice heart were harvested and digested. Cardiomyocytes (CMs) and non-cardiomyocytes (non-CMs) were analyzed against mCherry via flow cytometry.

FIG. 4F shows livers of mice as from FIG. 4E harvested, homogenized, and analyzed by flow cytometry for presence of mCherry expression. FIG. 4G shows left and right ventricular and atrial expression, in CMs and non-CMs, following iv (tail vein) injection with 3E10 viral genomes AAVExo-mCherry or 4.41E10 AAV-mCherry viral genomes.

FIGS. 5A-5C show AAVExo has high resistance to neutralizing antibodies in vitro using AC16 human ventricular cardiomyocyte cells. A) Dilutions of IVIg were mixed with either standard AAV6.mCherry or AAV6Exo.mCherry. After incubation for 30 mins, mixtures were added to AC16 cells and analyzed three days later using Flow Cytometry (A) and confocal fluorescence imaging (B) were performed (Shown are representatives of 4 biological replicates). C) Flow cytometry data compilation was shown. n=4.

FIGS. 6A-6V show AAVExo has high resistance to neutralizing antibodies in vitro using iPS-CM. A) Dilutions of IVIg were mixed with either standard AAV6.mCherry or AAV6Exo.mCherry. After incubation for 30 mins, mixtures were added to iPS cardiomyocytes and three days later iPS-CM were imaged by confocal fluorescence microscopy (C). After imaging, cells were stained with SERPA (marker for iPS cardiomyocyte) antibody and analyzed by Flow Cytometry (A). Flow cytometry data compilation was shown (B). Representative of three biological replicates is shown.

FIGS. 7A-7D show AAVExo has stronger transduction efficiency and resistance to Nab in vivo. A) Nude mice were preinjected with PBS or IVIg via i.p. 24 hours prior to AAV/AAVExo administration. Equal amounts of AAV or AAVExo was directly injected to myocardium with 5E¹⁰ g.c.p. Mice were imaged with IVIS Bioluminescence after 4 weeks, and Intensity of bioluminescence from the area of chest/heart was quantified (C). (*p=0.004). B) Ex vivo imaging confirmed AAVExo has stronger transduction efficiency and resistance to Nab. At 4-week point, heart and liver were collected and imaged with bioluminescence. Heart, liver and brain from plain mice and IVIg mice with treatment of saline, AAV or AAVExo were imaged and quantified (D).

FIGS. 8A-8C show AAV9Exo-SERCA2a improved cardiac functions in absence or presence of Nab in a mouse model with MI. Nude mice were pre-injected with IVIg (1 mg/animal) or saline via i.p. 24 hours before MI surgery. Same titer of AAVExo, AAV (1E¹¹ g.c.) or saline was intramyocardially injected nearby ischemic area immediately after surgery. After 2 weeks (A) and 4 weeks (B) ejection fraction and fractional shortening were measured by echocardiography. Mice number of each condition was indicated in left panel of (A). p: ****<0.0001, ***<0.005, **<0.01 *<0.05. C) Mice left ventricle wall thickness and movement was recorded by M mode of echocardiography.

FIG. 9 shows an alternative nonlimiting example of a method flow chart of AAVExo Purification (left) and separation of AAVExo from AAVs using iodixanol density gradiant (right). A sharp white band corresponding to AAVExo was found in an approximately 20% iodixanol layer.

FIGS. 10A-10C show separation of AAVExo from free AAV in accordance with aspects of the present disclosure.

FIGS. 11A-11D demonstrate AAVExo containing AAV capsids, expression of AAV capsid proteins, and specific exosomal surface markers, in accordance with aspects of the presend disclosure.

FIG. 12 shows NAbs in serum of NAb pre-injected nude mice inhibit AAV transduction in vitro as effectively as Abs naturally present in the serum of wild-type mice. Nude or WT mice were intravenously (tail vein) injected with equal titers of AAV9Exo-FLuc or AAV9-FLuc. 24 h prior to AAVExo/AAV administration, nude mice were IP injected with NAbs (NAb+). Animal sera collected from NAb pre-injected mice and WT mice were diluted (1:2), mixed with AAV9-FLuc, incubated for 30 min at 37 degrees C., and then added to HEK293T cells. Two days later, transduction efficiency was analyzed by quantifying luciferase activity. Error bars represend SD. N=4.

FIG. 13 shows AAV empty capsids allow for vector delivery in the presence of NAbs in vivo. FIG. 13A shows the experimental design, in which NAb+ and NAb− nude mice were injected iv with equal titers of AAV9-FLuc (3E11 viral genomes). 30 minutes before AAV9 administration, one group of NAb+ mice received formulation of AAV9 empty capsids (3E11 viral genomes). In FIG. 13B, top panel, bioluminsecent images show NAb− ad NAb+ mice transfected mice 6 weeks post-injection of equal titers of AAV9-FLuc in the presence or absence of empty AAV9 capsids. Bottom panel shows ex vivo imaging of hearts from NAb− and NAb+ mice transfected with equal titers of AAV9-FLuc in the presence of absence of empty AAV9 capsids.

FIG. 14 shows cellular trafficking of HEK293 cell-derived exosomes.

### DETAILED DESCRIPTION OF THE INVENTION

This disclosure relates to a method of making isolated exosomes containing an adeno-associated viral (AAV) vector, including disposing a suspension on an iodixanol gradient, wherein the suspension includes exosomes containing AAV vectors and the iodixanol gradient includes a higher layer of between 20% and 30% w/v iodixanol solution, an intermediate layer of between 30% and 50% w/v iodixanol solution, and a lower layer of between 50% and 70% w/v iodixanol solution, and centrifuging the iodixanol gradient at between 180,000 g and 300,000 g for at least 2 hours. In some embodiments, the method further includes, before disposing the suspension on the iodixanol gradient, forming a sucrose suspension by suspending exosomes containing AAV vectors in a 30% w/v solution of sucrose in deuterium oxide and centrifuging the first suspension at between 80,000 g and 120,000 g for at least 70 min to form a fraction of exosomes containing AAV vectors, washing the fraction, and suspending the fraction in a solution that does not contain sucrose.

In some examples, cells in which the exosomes containing AAV vectors were produced include 293T cells, Per.C6 cells, AGE1.CR cells, AGE1.HN cells, KG-1 cells, human umbilical vein endothelial cells (HUVECs), embryonic stem (ES) cells from humans, human mesenchymal stem cells (MSCs), human fibroblasts, human keratinocytes, human hematopoietic progenitor cells, human cKit+ stem cells, human cardiosphere-derived cells, HeLa cells, induced pluripotent cells, K562 cells, Caco-2 cells, human T-cells, human dendritic cells, human smooth muscle cells, or endothelial cells derived from vascular origin. In other examples, the lower layer includes 60% w/v iodixanol solution, the intermediate layer includes 40% w/v iodixanol solution, and the higher layer includes 25% w/v iodixanol solution. Further examples include centrifuging the iodixanol gradient at 250,000 g, or between 180,000 g and 200,000 g, or centrifuging the iodixanol gradient for at least 3 hours.

Still further examples include collecting a final fraction from the iodixanol gradient after centrifuging the iodixanol gradient, wherein the final fraction includes a highest portion of the first layer and the portion is up to one half volume of the higher layer. In other examples the portion is up to one quarter volume of the higher layer. In yet other examples the portion is up to one eighth volume of the higher layer. In still other examples, more than 95% of AAV vector genome copies present in the final fraction are in exosomes. In other embodiments, the AAV vectors are AAV1, AAV2, AAV6, or AAV9 vectors.

In another aspect, disclosed is a method of transfecting a cell, comprising contacting the cell with exosomes containing an adeno-associated viral (AAV) vector, wherein a method for making the exosomes containing an AAV vector includes disposing a suspension on an iodixanol gradient, wherein the suspension includes exosomes containing AAV vectors and the iodixanol gradient includes a higher layer of between 20% and 30% w/v iodixanol solution, an intermediate layer of between 30% and 50% w/v iodixanol solution, and a lower layer of between 50% and 70% w/v iodixanol solution, and centrifuging the iodixanol gradient at between 180,000 g and 300,000 g for at least 2 hours. In some embodiments, the method further includes, before disposing the suspension on the iodixanol gradient, forming a sucrose suspension by suspending exosomes containing AAV vectors in a 30% w/v solution of sucrose in deuterium oxide and centrifuging the first suspension at between 80,000 g and 120,000 g for at least 70 min to form a fraction of exosomes containing AAV vectors, washing the fraction, and suspending the fraction in a solution that does not contain sucrose.

In some examples, cells in which the exosomes containing AAV vectors were produced include 293T cells, Per.C6 cells, AGE1.CR cells, AGE1.HN cells, KG-1 cells, human umbilical vein endothelial cells (HUVECs), embryonic stem (ES) cells from humans, human mesenchymal stem cells (MSCs), human fibroblasts, human keratinocytes, human hematopoietic progenitor cells, human cKit+stem cells, human cardiosphere-derived cells, HeLa cells, induced pluripotent cells, K562 cells, Caco-2 cells, human T-cells, human dendritic cells, human smooth muscle cells, or endothelial cells derived from vascular origin. In other examples, the lower layer includes 60% w/v iodixanol solution, the intermediate layer includes 40% w/v iodixanol solution, and the higher layer includes 25% w/v iodixanol solution. Further examples include centrifuging the iodixanol gradient at 250,000 g, or between 180,000 g and 200,00 g, or centrifuging the iodixanol gradient for at least 3 hours.

Still further examples include collecting a final fraction from the iodixanol gradient after centrifuging the iodixanol gradient, wherein the final fraction includes a highest portion of the first layer and the portion is up to one half volume of the higher layer. In other examples the portion is up to one quarter volume of the higher layer. In yet other examples the portion is up to one eighth volume of the higher layer. In still other examples, more than 95% of AAV vector genome copies present in the final fraction are in exosomes. In other embodiments, the AAV vectors are AAV1, AAV2, AAV6, or AAV9 vectors.

In a particular aspect, disclosed is a method of making isolated exosomes containing an adeno-associated viral (AAV) vector, including forming a sucrose suspension by suspending exosomes containing AAV vectors in a 30% w/v solution of sucrose in deuterium oxide and centrifuging the first suspension at 100,000 g for at least 70 min to form a fraction of exosomes containing AAV vectors, washing the fraction, and suspending the fraction in a solution that does not contain sucrose, forming a suspension, disposing a suspension on an iodixanol gradient, wherein the suspension comprises exosomes containing AAV vectors and the iodixanol gradient comprises a higher layer of 25% w/v iodixanol solution, an intermediate layer of 40% w/v iodixanol solution, and a lower layer of 60% w/v iodixanol solution, centrifuging the iodixanol gradient at 250,000 g for at least 3 hours, and collecting a final fraction from the iodixanol gradient after centrifuging the iodixanol gradient, wherein the final fraction includes a highest portion of the first layer and the portion is up to one eighth volume of the higher layer, wherein the AAV vectors are AAV9 vectors and cells in which the exosomes containing AAV vectors were produced comprise 293T cells.

In yet another aspect, disclosed is a suspension including exosomes containing adeno-associated viral (AAV) vectors, wherein 95% of more of AAV vector genome copies present in the suspension are in exosomes. In one embodiment, the AAV vectors are AAV1, AAV2, AAV6, or AAV9 vectors. In a particular example, the AAV vectors are AAV9 vectors.

Exoosmes are double membrane-bound biological nanovesicles, actively secreted by almost all cell types known. Typically, exosomes may be between approximately 50-200 nm in diameter. AAVs are non-pathogenic, non-integrating and non-mutagenic vectors capable of effecting long-trem gene delivery. AAVs can be cardiotropic and can deliver genes to non-dividing and slowly dividing cells such as cardiomyocytes.

Isolated AAVExo are AAVExo that exist in a sample, suspension, or population that have been isolated from AAV vectors, particles, or genetic material not contained in AAVExo. A sample, suspension, or population contains a number of copies of an AAV vector genome, or AAV genome copy. Some AAV vector genome copies may be contained within AAVExo, whereas others may be contained in AAV that are not contained within exosomes. A proportion of AAV genome copies in a sample, suspension, or population, that are in AAVExo relative to the total number of AAV genome copies in said sample, suspension, or population inclusive of AAV genome copies present in exosomes and not present in exosomes, can be measured as disclosed herein. If the number of AAV genome copies present in exosomes is more than 90% of all AAV genome copies in a sample, suspension, or population, then the AAVExo therein are isolated exosomes containing adeno-associated viral vectors.

In other examples, the AAV genome copy of AAVExo may be less than 90%, in which case they would not be considered isolated, but could still be prepared in accordance with the present disclosure. For example, the AAV genome content of AAVExo may be between 85% and 90%, 80% and 85%, 80% and 90%, 75% and 80%, 75% and 85%, 75% and 90%, or between 50% and 90%. In examples of isolated AAVExo, the proportion of AAV genome copies present in exosomes in a sample, suspension, or population may be between 90% and 95%, between 95% and 100%, between 90% and 92%, between 92% and 95%, between 92% and 97%, between 95% and 97%, or between 90% and 97%, or within any derivable range therein.

In some embodiments, a method of making isolated exosomes includes disposing a sample, suspension, or population of AAVExo on a solution containing iodixanol. Iodixanol may be dissolved in a solution, typically water, typically with a buffer for maintaining physiological pH (approximately 7.4) and physiological concentration of sodium chloride (approximately 150 mM). Solutions of iodixanol with different percentages by weight of iodixanol will have different densities. For example, a 25% w/v solution of iodixanol will have a density of 1.137 g/ml, a 40% w/v solution of iodixanol with have a density of 1.125 g/ml, and a 60% w/v iodixanol solution will have a density of 1.320 g/ml. An iodixanol solution may be a homogeneous solution of a single density or may consist of a gradient. A gradient is a combination of layers of iodixanol solutions with different densities from each other. In a vertically held centrifugation tube, a higher density iodixanol solution may be at the bottom of the tube, and a lower density iodixanol solution may be farther away from the bottom than solutions with higher density.

An iodixanol solution may have two, three, or more layers of different densities of iodixanol, forming an iodixanol gradient. As referred to herein, a lower layer of iodixanol in an iodixanol gradient is a layer with a higher density than and therefore closer to the bottom of the tube than at least two other layers, although it is possible that another layer of still higher density may be below a so-called lower layer in a centrifuge tube. Similarly, as referred to herein, a higher layer of iodixanol in an iodixanol gradient is a layer with a lower density than and therefore farther from the bottom of the tube than at least two other layers, although it is possible that another layer of still lower density may be above a so-called higher layer in a centrifuge tube. Finally, an intermediate layer has a density higher than the density of a higher layer but lower than the density of a lower layer and therefore exists between said higher and lower layers, although still other layers may exist between an intermediate layer and a higher layer and between an intermediate layer and a higher layer. Thus, among a higher layer, an intermediate layer, and a lower layer, no layer must be contiguous with any other layer, although all three layers may be contiguous.

The volume of each layer may differ from any one or two other layers, or two or more layers may have the same volume. For example, a higher layer, and intermediate layer, and a lower layer may each independently be anywhere from between 0.5 ml to 6 ml in volume.

In turn, portions of each layer may be drawn off of or taken from an iodixanol gradient layer. And each such portion may constitute any chosen percentage by volume of the layer from which it was drawn or taken. A portion that is nearest the top of a layer is referred to as a highest portion, in that it is farthest from the bottom of the centrifuge tube holding the iodixanol gradient, and a lowest portion would therefore mean a portion that is nearest the bottom of a layer. An intermediate portion would be between a highest portion and a lowest potion. Another portion or portions may exist between an intermediate portion and a highest portion or between an intermediate portion and a lowest portion. Thus, a lowest portion need not be contiguous with an intermediate or highest portion, and a highest portion need not be continuous with an intermediate portion. In some examples, a portion may include a highest portion of a first layer and a lowest portion of a second layer that is higher than the first layer.

When a sample containing AAVExo and possibly AAV not contained in exosomes (referred to herein as free AAV) is disposed on an iodixanol gradient and the iodixanol gradient is centrifuged, depending on the density of the highest layer of the iodixanol gradient, the AAVExo and free AAV get pulled toward the bottom of the gradient. Free AAV travels faster through the gradient than AAVExo. Thus, centrifuging a mixture of AAVExo and free AAV on an iodixanol gradient may separate AAVExo from free AAV. Various factors influence the degree of separation between AAVExo and free AAV that may be obtained by this method, including the number, density, and volume of layers in the gradient and the rate and duration of spinning.

For example, a higher layer of an iodixanol gradient may be from between 20% to 30% w/v iodixanol and have a volume of from between 0.5 ml to 6 ml. In some examples, a higher layer may be 21% w/v, 22% w/v, 23% w/v, 24% w/v, 25% w/v, 26% w/v, 27% w/v, 28% w/v, or 29% w/v iodixanol, or anywhere within a range between any two of the foregoing. In a particular embodiment, a higher layer is 25% w/v iodixanol. A higher layer may be 0.5 ml, 1 ml, 1.5 ml, 2 ml, 2.5 ml, 3 ml, 3.5 ml, 4 ml, 4.5 ml, 5 ml, 5.5 ml, or 6 ml in volume, or anywhere within a range between any two of the foregoing. In a particular embodiment, a higher layer may have a volume of 5 ml.

An intermediate layer of an iodixanol gradient may be from between 30% to 50% w/v iodixanol and have a volume of from between 0.5 ml to 6 ml. In some examples, an intermediate layer may be 31% w/v, 32% w/v, 33% w/v, 34% w/v, 35% w/v, 36% w/v, 37% w/v, 38% w/v, 39%, 40%, 41%, 42%, 43%, 44%, 45%, 46%, 47%, 48%, or 49% w/v iodixanol, or anywhere within a range between any two of the foregoing. In a particular embodiment, an intermediate layer is 40% w/v iodixanol. An intermediate layer may be 0.5 ml, 1 ml, 1.5 ml, 2 ml, 2.5 ml, 3 ml, 3.5 ml, 4 ml, 4.5 ml, 5 ml, 5.5 ml, or 6 ml in volume, or anywhere within a range between any two of the foregoing. In a particular embodiment, an intermediate layer may have a volume of 3 ml.

A lower layer of an iodixanol gradient may be from between 50% to 70% w/v iodixanol and have a volume of from between 0.5 ml to 6 ml. In some examples, an intermediate layer may be 51% w/v, 52% w/v, 53% w/v, 54% w/v, 55% w/v, 56% w/v, 57% w/v, 58% w/v, 59%, 60%, 61%, 62%, 63%, 64%, 65%, 66%, 67%, 68%, or 69% w/v iodixanol, or anywhere within a range between any two of the foregoing. In a particular embodiment, an intermediate layer is 40% w/v iodixanol. An intermediate layer may be 0.5 ml, 1 ml, 1.5 ml, 2 ml, 2.5 ml, 3 ml, 3.5 ml, 4 ml, 4.5 ml, 5 ml, 5.5 ml, or 6 ml in volume. In a particular embodiment, an intermediate layer may have a volume of 1 ml, or anywhere within a range between any two of the foregoing.

An iodixanol gradient may be centrifuged at anywhere from between 180,000 g and 300,000 g. For example, it can be centrifuged at 180,000 g, or 190,000 g, or 200,000, or 210,000, or 220,000, or 230,000, or 240,000, or 250,000, or 260,000, or 270,000, or 280,000, or 290,000, or 300,000 g, or anywhere within a range between any two of the foregoing. In an example, an iodixanol gradient may be centrifuged at 250,000 g, or between 180,000 g and 190,000 g. And an iodixanol gradient may be centrifuged for an hour or longer, 90 min or longer, 2 hours or longer, 2.5 hours or longer, 3 hours or longer, 3.5 hours or longer, 4 hours or longer, 4.5 hours of longer, 5 hours or longer, 5.5 hours or longer, 6 hours or longer, 6.5 hours or longer, 7 hours or longer, or aby other range, from any of the foregoing up to 12 hr, or up to 14 hr, or up to 16 hr, or up to 18 hr, or up to 20 hr, or up to 22 hr, or up to 24 hr, or anywhere within a range between any two of the foregoing. In an embodiment, an iodixanol gradient may be centrifuged for 3 hr or longer.

After an iodixanol gradient on which a sample, suspension, or population of AAVExo is disposed on an iodixanol gradient and the iodixanol gradient is centrifuged, the AAVExo become isolated in a portion or portions of a layer. A final fraction may be collected from a layer and the final fraction may contain isolated AAVExo. For example, the final fraction may include a highest portion of a higher layer. The highest portion of the higher layer may include not more than 90%, 85%, 80%, 75%, 65%, 60%, 55%, 50%, 45%, 40%, 35%, 30%, 25%, 20$, 15%, 12.5%, 10% 7.5%, 5%, or 2.5% of the higher layer by volume, or anywhere within a range between any two of the foregoing. In an embodiment, the final fraction includes a highest portion that is 12.5% by volume of the higher layer. In another embodiment, the final fraction includes a highest portion that is 25% by volume of the higher layer.

A relative number of AAV genome copy number in a final fraction, or portion, or layer, that is due to AAVExo AAV genome copy number and free AAV genome copy number may be calculated by performing methods disclosed herein on a sample containing AAVExo, a sample containing a mixture of exosomes that do not contain AAV and free AAV, and a sample containing free AAV and no exosomes. If the iodixanol gradient centrifugation steps are performed using a sample, suspension, or population of AAVExo and also performed on a mixture of exosomes and free AAV and on free AAV, portions of layers collected, and the AAV genome copy of different layers compared, the proportion of AAV genome copy in a portion containing AAVExo that is attributable to AAV contained in the AAVExo and free AAV can be determined. Free AAV are more dense and ay therefore travel farther down an iodixanol gradient during centrifugation than exosomes that do not contain AAV. AAVExo have a density between exosomes lacking AAV and free AAV.

Thus, genome copy of portions of layers of an iodixanol gradient on which only free AAV was disposed is attributable to AAV genome copy from free AAV. Because an AAVExo sample may contain free AAV, portions of a centrifuged iodixanol gradient on which AAVExo had been disposed and which contain AAV genome can be compared to comparable portions of a centrifuged iodixanol gradient on which free AAV had been disposed. If a portion of both such samples has AAV genome copies, differences between the relative number of copies may be attributable to differences in free AAV genome copies (shown by the free AAV portion) and AAV present in AAVExo in (shown by the difference between the AAV genome copy of the free AAV portion and the AAV genome copy of the AAVExo portion). For certain portions, AAV genome copy levels from the free AAV iodixanol gradient may be less than 10% of the AAV genome copy levels of a corresponding portion of an AAVExo iodixanol gradient. Such portions contain isolated AAVExo. For some portions, AAV genome copy levels from the free AAV iodixanol gradient may be less than 10%, 9%, 8%, 7%, 6%, or 5% of the AAV genome copy levels of a corresponding portion of an AAVExo iodixanol gradient. AAV genome copy number of a portion can be determined by quantitative PCR by well-known methods and as disclosed herein.

To further make and use isolated AAVExo, be removing contaminating free AAV from a sample, suspension, or population of AAVExo, such sample, suspension, or population may be centrifuged on a sucrose cushion, before being disposed on an iodixanol gradient. After a suspension, sample, or population has been obtained, it may be disposed in a solution containing sucrose. Sucrose may be in water, or in a preferred embodiment in deuterium oxide. The sucrose solution may further contain a buffer to maintain the sample at physiological pH (approximately 7.4) and sodium chloride at physiological levels (approximately 150 nM). The sucrose solution may be 20%, 25%, 30%, 35%, or 40% w/v sucrose. In a preferred embodiment, it is 30% w/v sucrose (which has a density of 1.210 g/ml). The AAVExo sample, suspension, or population may be centrifuged in the sucrose cushion at 80,000 g, 90,000 g, 100,000 g, 110,000 g, 120,000 g, or within any range derivable therefrom between any two of the foregoing. In an embodiment, it may be centrifuged at 100,000 g. It may be centrifuged for anywhere for 60 min or longer, 65 min or longer, 70 min or longer, 75 min or longer, 80 min or longer, 85 min or longer, or 90 min or longer, or within any range derivable therefrom between any two or more of the foregoing. In an example, it may be centrifuged for 70 min or longer.

After centrifugation, the remaining sucrose suspension, containing the AAVExo, may be collected, leaving the centrifuged pellet, and the sucrose suspension may be diluted with buffered saline to decrease the concentration of sucrose, then centrifuged again to pellet AAVExo. For example, the diluted sucrose suspension may be centrifuged for 70 min at 100,000 g. The result of this centrifugation step is the formation of a pellet containing AAVExo which, following separation of the centrifuged diluted sucrose supernatant, may be resuspended in an aqueous buffer, such as phosphate buffered saline. The resulting resuspension of AAVExo may be disposed on an iodixanol gradient for continuation of isolation of AAVExo.

For example, before disposing the suspension on the iodixanol gradient, forming a sucrose suspension by suspending exosomes containing AAV vectors in a 30% w/v solution of sucrose in deuterium oxide and centrifuging the first suspension at between 80,000 g and 120,000 g for at least 70 min to form a fraction of exosomes containing AAV vectors, washing the fraction, and suspending the fraction in a solution that does not contain sucrose.

AAVExo may be made by known methods disclosed in publicly available literature and known to skilled artisans in the relevant field. AAVExo may be made in cells engineered to produce AAV vectors and which also produce exosomes, leading to the production of exosomes that contain AAV. Examples of cells that can be engineered to produce a sample, suspension, or population of AAVExo include 293T cells, Per.C6 cells, AGE1.CR cells, AGE1.HN cells, KG-1 cells, HUVECs, embryonic stem (ES) cells from humans, human mesenchymal stem cells (MSCs), Human fibroblasts from different sources (such as from skin, foreskin, epithelial cells, hearts etc.), human keratinocytes, human hematopoietic progenitor cells (e.g. CD34+, CD133+) from blood and bone marrow, human cKit+ stem cells from blood, heart and other organs, human cardiosphere-derived cells from pediatric and adult sources, HeLa cells and cells derived from HeLa cells, induced pluripotent cells from different sources, K562 cells, Caco-2 cells, human T-cells, human dendritic cells, human smooth muscle (e.g., AoSMC, PASMC, CASMC, UASMC, etc.) and endothelial cells (e.g., HCAECs, HAECs, HPAECs, HDMVECs, etc.) derived from vascular origin. In an example, cells in which AAVExo were produced and from which they are obtained are 293T cells.

The methods disclosed herein may be used for isolating AAVExo containing AAV of any of different serotypes. For example, AAVExo may contain AAV1, AAV2, AAV3, AAV4, AAVS, AAV6, AAV7, AAV8, AAV9, or AAV10. In some examples, AAVExo contain AAV1, AAV2, AAV6, or AAV9. In an example, AAVExo contain AAV9. AAV in AAVExo may further be designed to carry any of various known payloads. For example, the AAV may code for translatable mRNA to be produced in a cell, or an siRNA molecule, or an RNA molecule designed to bind to a cellular target. The method described herein for making and isolating AAVExo is not limited to a particular payload an AAV contained in an AAVExo carries.

A sample, suspension, or population of AAVExo may be separated from cells which were used to produce such AAVExo according to known methods. For example, cell medium in which such cells were growing to produce such AAVExo may be collected, then subjected to serial centrifugation steps, followed by discarding a precipitated pellet after each step. Sequential centrifugation steps may include one of more of the following steps: centrifugation at 100 g, 200 g, 500 g, 2,000 g, 10,000 g, 100,000 g, and 120,000 g sequenced in steps of increasing g (or optionally with a centrifugation step at a given g repeated one or more times in a row), and collecting the supernatant after each step and centrifuging the collected supernatant in the next step. Other steps may include centrifugation at 1,000 g, 1,500 g, 2,500 g, 5,000 g, 7,500 g, 15,000 g, 25,000 g, 50,000 g, 75,000 g, 90,000 g, and 110,000 g, again centrifuging the supernatant collected after a previous centrifugation step at the same or a lower g. Following sequential centrifugation steps, the remaining supernatant may be used in subsequent sucrose cushion or iodixanol gradient steps. Successive centrifugation steps may be for 10 min, 15 min, 20 min, 25 min, 30 min, 35 min, 40 min, 45 min, 50 min, 55 min, 60 min, 65, min, 70 min, 75 min, 80 min, 85 min, 90 min, 95, min, 100 min, 105 min, 110 min, 120 min, or longer. In an example 5 such steps may include 10 mon centrifugation at 200 g, 10 min centrifugation at 500 g, 15 min centrifugation at 2,000 g, 20 min centrifugation at 10,000 g, and 90 min centrifugation at 120,000 g. Additional centrifugation steps may be included between any two of these foregoing steps, any such step or steps may be for a different g or duration from the foregoing example or, in some examples, may be omitted altogether. Some examples may include four successive centrifugation steps, at 500 g, 2,000 g, 10,000 g, and 100,000 g, for example. Other options may also be selected applicable to various embodiments to suit a given application. A skilled person would appreciate that various permutations of numbers of steps of various durations and different g forces applied could be adopted for use in accordance with a method as disclosed herein.

An isolated AAVExo may be administered to a subject, such as a human or nonhuman animal, by any of various means suitable to a purpose for which it is being administered, such as to treat a disease, condition, illness, or disorder, meaning to lessen, ameliorate, or shorten untoward symptoms of pathological processes. An AAVExo may be injected intravenously, intramuscularly, intraperitoneally, intracranially, intracerebroventricularly, intracardially, subcutaneously, intravitreously, intramyocardially, or otherwise. Isolated AAVExo may be injected directly into an organ where cellular transfection is desired, or into general circulation or otherwise for dissemination throughout a body, or so as to reach an intended organ or organs. As skilled artisans would understand, any solvents, excipients, or other components of a composition including isolated AAVExo may be included as would be appropriate to facilitate delivery of AAVExo to the intended site or sites.

AAVExo in a sample, suspension, or population of isolated AAVExo may be from between 50-200 nm in diameter. In some examples, they may be between 90-110 nm in diameter. In some examples they may be between 110 nm and 130 nm, or between 130 nm 150 nm, or between 150 nm and 175 nm, or between 175 nm and 200 nm, or between 70 nm and 90 nm, or between 50 nm and 70 nm in diameter. In still other examples they may be between 75 nm and 125 nm in diameter. Or they may be of a diameter within any range derivable therefrom between any two of the foregoing.

### EXAMPLES

The following examples are intended to illustrate particular embodiments of the present disclosure, but are by no means intended to limit the scope thereof.

Although preferred embodiments have been depicted and described in detail herein, it will be apparent to those skilled in the relevant art that various modifications, additions, substitutions, and the like can be made without departing from the spirit of the present disclosure and these are therefore considered to be within the scope of the present disclosure as defined in the claims that follow.

Methods

Cell Culture: Human (HEK) 293T cells were obtained from the American Type Culture Collection (Manassas, Va.) and cultured in high glucose Dulbecco's modified Eagle's medium (Life Technologies, Grand Island, N.Y.) supplemented with 10% fetal bovine serum (Thermo Fisher Scientific, US) and 1% streptomycinpenicillin (Corning, Manassas, Va.) in a humidified atmosphere supplemented with 5% CO2 at 37° C. AC16 cell line obtained from the American Type Culture Collection (Manassas, Va.) and cultured in the Dulbecco's modified Eagle's medium with same supplements as that of 293T cell culture. IPS-derived cardiomyocytes were generated and kindly offered by Dr. Delaine Ceholski. HEK293T cells naturally secrete AAV-containing exosomes.

AAV and AAVExo production and purification: AAVs were produced by double transfection of HEK-293T cells as described previously. Briefly, cells were cultured in the T175 flask with culture medium. When achieved 60-70% confluency, cell culture medium was replaced with transfection reagent which was made by mixing 50 ug of the helper plasmid, 17 ug of the transgene plasmid and 233 ul of polyethylenimine (1 mg/ml, linear, MW 25,000; cat. no.: 23966; Polysciences, Warrington, Pa.) in DMEM supplemented with 2% FBS and streptomycin-penicillin. The cells were collected 3 days later at 300 g for 10 minutes (cell-free supernatant saved for AAVExo purification) and resuspended in 10 ml of lysis buffer (150 mmol/1 sodium chloride, 50 mmol/1 Tris-HCl pH 8.5), subjected to three freeze-thaw cycles and treated with 1,500 u of Benzonase Nuclease (cat. no. E1014; Sigma-Aldrich) in the presence of 1 mmol/1 magnesium chloride for 1 hour at 37° C. Cellular debris was removed by centrifugation for 10 minutes at 5,000 g (Sorval RC-4, LH-4000 rotor). The virus was purified by a four-step iodixanol gradient centrifugation (7.3 ml of 15%, 4.9 ml of 25%, 4 ml of 40%, and 4 ml of 60% iodixanol (Optiprep; Sigma-Aldrich, cat. no. D1556) were overlayed with 10 ml of cell lysate in lysis buffer) in a 70Ti rotor (Beckman Coulter, Brea, Calif.) at 68,000 r.p.m. for 1 hour using OptiSeal Polyallomer Tubes (cat. no.: 361625; Beckman Coulter). The 40-60% interphase of the gradient was collected and the buffer was exchanged using VIVASPIN 20 column with 100,000 MWCO (Sartorius, Goettingen, Germany) against lactated Ringer's solution.

AAVExo were purified from cell culture medium by a combination of ultracentrifugation, sucrose cushion and Optiprep density gradient (Sigma-Aldrich, St Louis, Mo). Specifically, cell-free supernatant was sequentially centrifuged at 2000 g and 10,000 g to remove cell debris and large vesicles. Supernatant from 10,000 g was ultracentrifuged under 100,000 g and crude exosome pellet was resuspended in PBS followed by 100,000 g (Beckman Coulter, Brea, Calif.) for 1 hour on a 30% sucrose-D20 solution. Purified exosomes were then loaded on top of a three-step iodixanol gradient (25%, 40% and 60%) and centrifuged at 250,000 g for 3 hours (Beckman Coulter, Brea, Calif.). One ml fractions from the top to the bottom of the gradient were collected. Fraction 4 that contained AAVExo was diluted in PBS and centrifuged at 100,000 g for 1 hour. AAVExo pellet was resuspended in PBS for in vitro and in vivo experiments. The titers of AAV and AAVExo were determined by qPCR using the SYBR Advantage qPCR Premix (Clontech, Mountain View, Calif.) with an Applied Biosystems (Carlsbad, Calif.) 7500 real-time PCR system with primers against the CMV sequence (Forward: 5′-TCAATTACGGGGTCATTAGTTC-3′; Reverse: 5′-ACTAATACGTAGATGTACTGCC-3′)

Electron microscopy: AAV-producing 293T cells were washed with PBS and fixed on the flask with 1% glutaraldehyde for 20 minutes. Then cells were gently scrapped off the flask and washed with PBS, followed by 1% osmium tetroxide for 40 minutes at room temperature. Cells were then embedded in epon resin (Electron Microscopy Sciences, Hatfield, Pa.). Thin and ultrathin sections were cut on an ultramicrotome (Leica Ultracut UCT) and stained with uranyl acetate and lead citrate. Samples were observed using Hitachi H7650 or S4300 microscopes operating at 30˜50 kV.

In vitro transduction: HEK-293T or AC16 cells were seeded on 24-well plate and cultured in DMEM with 10% FBS and penicillin/streptomycin. Cells were ready for AAVExo or AAV infection when reached ˜70% confluency. IPS-CM cells were maintained in RPMI supplemented with B27 when ready for infection. Dilutions of IVIg from 0.5-4 mg/ml or equal volume of PBS were mixed with AAVExo or AAV for 30 minutes at 37° C. and then added to cell culture. Three days later cells were ready for flow cytometry analysis.

In vivo gene-delivery experiment: All animal experiments were approved by Icahn School of Medicine at Mount Sinai Institutional Animal Care and Use Committees in accordance with and was in compliance with institutional and governmental regulations (PHS Animal Welfare Assurance A3111-01). Nude mice (Nu/J, 8-10 weeks, weight, 25˜30 g) were purchased from Jackson Laboratory (Bar Harbor, Me.). Tail vein injection of IVIg: For tail vein injections of IVIg, mice were placed into a restrainer, and the tail was warmed for 30 seconds by light and wiped by 70% isopropyl alcohol pads. A 200 μl volume of IVIg (1 mg per animal in saline) was slowly injected into a lateral tail vein before gently finger clamping the injection site until bleeding stopped.

Myocardial infarction and intramyocardial injection: Myocardial infarction was induced by LAD coronary artery ligation in mice. Briefly, mice were anaesthetized intraperitoneally with ketamine (0.065 mg g-1 body weight), acepromazine (0.001 mg g-1 body weight) and xylazine (0.013 mg g-1 body weight). After thoracotomy, LAD ligation was performed with a 7-0 silk suture 3-4 mm from the tip of the left auricle. The successful performance of LAD ligation was verified by visual inspection of the color of the apex. Then a volume of 50 μl AAVExo or AAV or saline was injected to 3˜4 sites around ischemic zone at left ventricle and apex. After injection the chest was closed with a 6-0 silk suture, and the skin was closed with 4-0 silk sutures. All mice were housed under identical conditions and were given water and soft food in following 3 days.

Bioluminescence imaging of firefly luciferase (FLuc) expression: Imaging was performed using an IVIS® Spectrum optical imaging system fitted with an SurgiVet Gas Anesthesia System (Caliper Life Sciences, Hopkinton, Mass.). Mice were anesthetized and then injected intraperitoneally with D-luciferin resuspended in PBS (150 μg/g body weight; Sigma). Post-injection mice were imaged for luciferase expression using IVIS100 charge-coupled device imaging system every 2 minutes until the FLuc signal reached a plateau. Data analysis for signal intensities and image comparisons were performed using Living Image® software (Caliper Life Sciences). To calculate total flux in photons/sec for each animal, regions of interest were carefully drawn around chest/heart region.

Ex-vivo organ luciferase assay: Four weeks post-AAVExo-/AAV-FLuc injection, mice were sacrificed and organs were harvested for analysis of FLuc levels. Mice were anesthetized and then injected intraperitoneally with D-luciferin 5-7 minutes before tissues were quickly removed from the animals, and immediately imaged for luciferase expression. Data analysis were performed using Living Image® software.

Echocardiography measurement: Mice were anaesthetized with SurgiVet Gas Anesthesia System for echocardiographic scanning. Transthoracic echocardiography was performed using a Visual Sonic Vevo 2100 Micro-Ultrasound Imaging system (FUJIFILM VisualSonics INC. 3080 Yonge Street, Suite 6100 Box 66, Toronto, Ontario, Canada) equipped with a MS550D transducer (40 MHz). Two-dimensional B mode movies were obtained in the long-axis view and M-mode images were obtained in the short-axis view. The heart rate was kept at 475±50 bpm during measurement. LV end-diastolic volume and LV endsystolic volume were measured from B mode for calculation of ejection fraction. LV end-diastolic internal diameter and LV end-systolic internal diameter were measured from M mode for calculation of fractional shortening.

Flow cytometry: Mouse cardiomyocytes and non-cardiomyocytes were obtained by enzymatic dissociation of the heart following standard perfusion procedures with modifications. Briefly, AAVExo- or control-injected mice were injected with heparin 15 min before heart excision and anaesthetized by isoflurane inhalation. Hearts were quickly removed from the chest and perfused with Ca2+-free solution containing collagenase type II (Worthington, Lakewood, N.J., USA). Ventricles were cut into small pieces and gently minced with a Pasteur pipette. Dissociated cells were transferred to a 50 ml Falcon tube and kept in Tyrode's solution at room temperature for 5-10 min. Ventricular cardiomyocytes settled on the bottom of the tube. Most non-cardiomyocyte cells were then collected without disturbing the cardiomyocyte layer for flow cytometric analysis. HEK-293T, AC16 or iPS-CM cells were harvested and resuspended in FACS buffer (PBS with 0.5% BSA and 2mM EDTA). IPS-CM cells were incubated with anti-SERPA antibody (PE/Cy7 anti-human CD172 a/b (SIRPα/β); Clone SE5A5, Biolegend, Cat:! 323808) on ice for 30 minutes in dark, washed with PBS and then resuspended in FACS buffer. DAPI was added before flow cytometry analysis (LSRII Cell Analyzer, BD Biosciences). Data was analyzed by FCS Express 5 Flow Cytometry analysis software.

Results

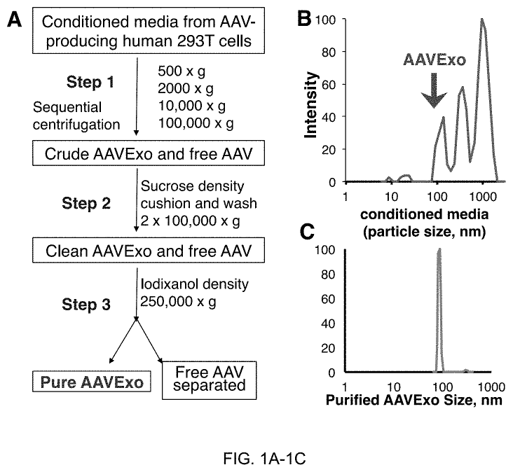

Strategies for purification of AAVExo with minimal contamination of free AAVs: 293T cells, widely used to generate AAVs secrete exosomes. To test whether they also secrete AAV-containing exosomes, 293T cells were transfected with standard plasmids used previously to produce double-stranded AAV-EGFP. Extracellular secretion of AAV9 was significantly higher compared to AAV1 and AAV2 (data not shown). Therefore, AAV9 was used in subsequent experiments. In addition to AAVExo, free AAV is released to culture medium as well. In an example for isolating AAVExo without significant contamination from free AAV, steps as shown in FIG. 1A were performed. Crude AAVExo plus co-pelleted free AAV were obtained in Step 1, and in some examples purified AAVExo separated from protein aggregates in Step 2 (as confirmed by DLS analysis). Steps 1 and 2 are exemplary and optional, as other steps may be performed, or parts or all of one or the other of these steps may be eliminated, and a resulting process may still be within methods disclosed and claimed herein. But as for the process as illustrated in FIG. 1A, Step 2 was not sufficient to separate free AAVs from AAVExo (qPCR data not shown). Therefore, Step 3 was performed with 25-60% iodixanol density gradient based on the flotation density of exosomes, AAVExo and free AAV. Purity of AAVExo was compared before and after three steps of isolation using dynamic light scattering (DLS). In raw conditioned medium population of AAVExo (˜100 nm) along with small and lager particles were observed (FIG. 1B), while after 3-step isolation clean population of AAVExo was obtained (FIG. 1C).

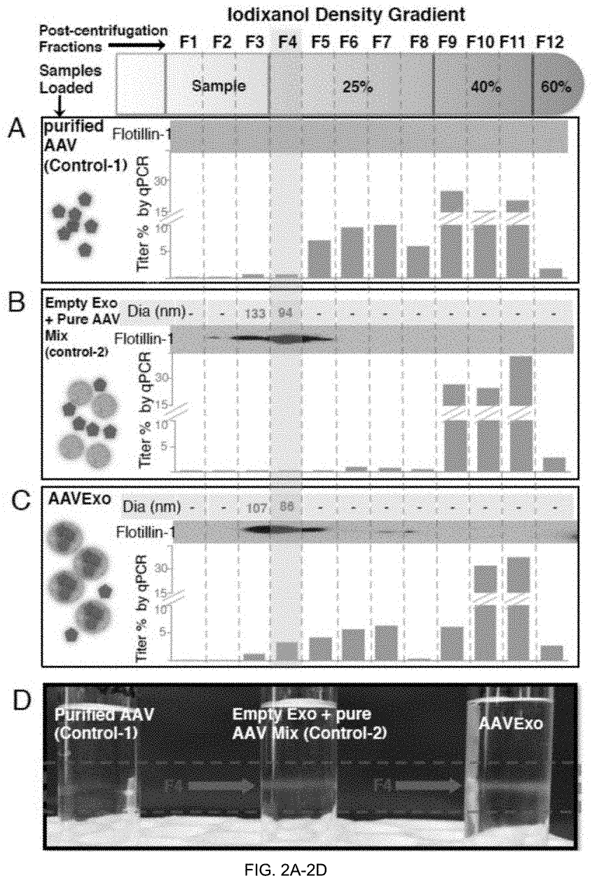

Purification efficiency was evaluated by including sophisticated controls. Purified free AAVs was loaded as control-1 (FIG. 2A) to demonstrate absence of free AAV in the AAVExo fraction. In addition, control-2 was included, which was a mixture of empty wild-type exosomes and purified free AAVs (pre-incubated for 1 h, at 37° C. before loading on gradient), to demonstrate that most free AAVs do not bind or stick to the surface of exosomes nonspecifically (FIGS. 2B and C). Control-1, control-2 and AAVExo had equal amount of AAVs in genome copy (g.c) number. Control-2 and AAVExo had equal amount of exosomes in protein level. After ultracentrifugation, we observed a white exosomes layer floating at ˜20% both in AAVExo and control-2 gradient, but not in control-1 gradient (FIG. 2D, arrows), which is consistent with reported density of exosomes. Separate fractions with equal volume were collected from the top, and the layer with white band was precisely collected as fraction 4 (F4). Each fraction was analyzed for presence of exosomes (1. size, by DLS; 2. exosomes marker protein, flotillin-1 by Western Blot (WB)), and for the g.c. number of AAV by qPCR (FIG. 2 A, B, C).

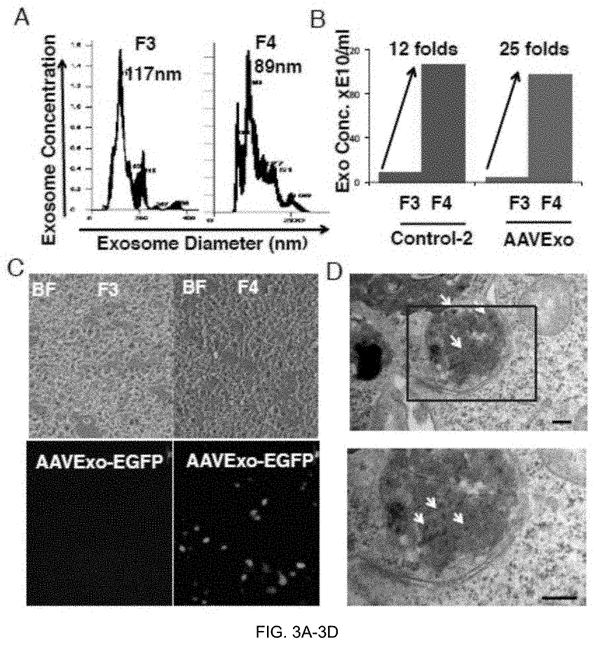

The DLS and WB data indicates that exosomes were primarily located in F3 and F4 (marked green). Further, significant level of AAV genome was detected in F3 and F4 of AAVExo, but not in controls, suggesting that the purification process successfully isolated the AAVExo from free AAVs secreted by 293T cells. As both F3 and F4 contained AAVExo, we analyzed them using Nanoparticle Tracking Analysis (NTA) to determine the size and quantity of exosomes. AAVExo had comparable sizes in F3 and F4 (FIG. 3A), while for quantity, F4 from both control-2 and AAVExo had ten- to twenty five-fold more exosomes than respective F3 (by particles/ml, FIG. 3B). Further, transduction efficiency of AAV9Exo-EGFP from F4 was significantly higher compared to F3 using 293T cells (FIG. 3C). Next using transmission electron microscopy (TEM) it was confirmed that AAVs packed in exosomes. AAV-producing 293T cells were fixed and embedded in resin, of which the sections were imaged by TEM. Within cytoplasm were observed exosomes in the multivesicular body, a membrane-bound structure that will fuse to plasma membrane and release exosomes to extracellular environment. Notably, viral particles were found within exosomes, as indicated by arrows in FIG. 3D. Therefore, AAVExo purified from F4 was relatively pure without significant contamination of free AAVs. AAVExo from F4 had higher transduction efficiency compared to F3. Thus, F4 was chosen as AAVExo for subsequent studies.

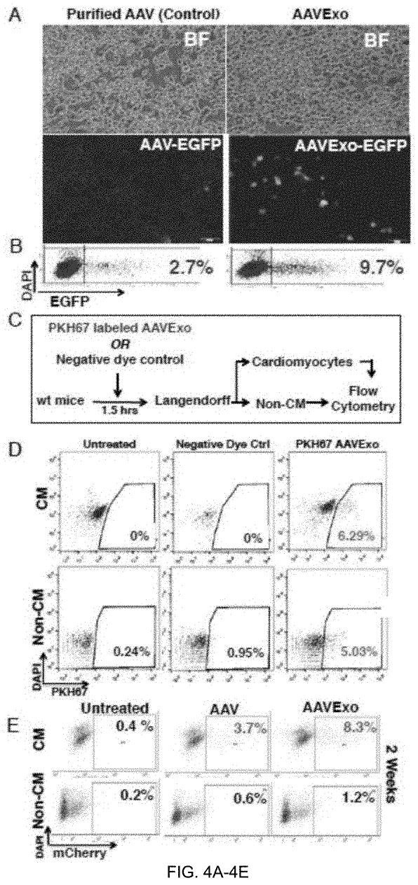

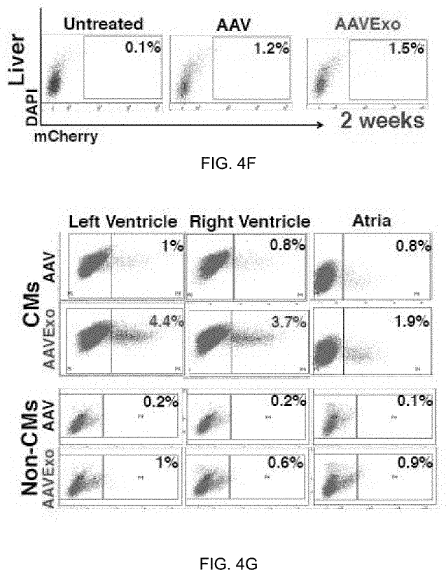

AAVExo has higher gene delivery efficiency compared to AAV: To compare the gene transfer efficiency of AAV and AAVExo, double-stranded AAV9-EGFP and AAV9Exo-EGFP were produced using the above protocol. Wild type 293T cells were treated with equal titer of AAV or AAVExo. Cells were analyzed by fluorescence microscopy (FIG. 4A) and flow cytometry analysis (FIG. 4B). The result demonstrated that in vitro AAVExo had significantly higher transduction efficiency than AAV. Exosomes can be rapidly internalized by recipient cells. Fast entry of vectors to recipient cells is beneficial to efficiency of gene delivery, analyzed AAVExo uptake by cardiac cells in vivo was analyzed. To trace the vector, AAVExo was fluorescently labeled with PKH67 before injected to mice myocardium. To rule out any free dye contamination, a negative dye control was added, where same amount of PKH67 was incubated with saline instead of AAVExo. The sample or control was washed to eliminate free dye before injected to mice heart. After 1.5 hours of injection, cardiomyocytes and non-myocytes were isolated using Langendorff perfusion system followed by flow cytometry analysis (FIG. 4C). Both cardiomyocytes and non-myocytes had AAVExo internalized (FIG. 4D), indicating that after intramyocardial injection AAVExo were taken up by cardiac cells within a short period of time. To determine the transduction efficiency of AAVExo in vivo, 4.4×10¹⁰ g.c. of AAV9ExomCherry or AAV9-mCherry were injected intramuscularly to the heart of C57BL/6 mice. The ventricular tissue was harvested at different time points and cardiomyocytes and non-cardiomyocytes were isolated using Langendorff perfusion system. Live cells were analyzed by flow cytometry for mCherry (FIG. 4E). Both at one week and two weeks (FIG. 4E), AAVExo-injected hearts had significantly higher mCherry-positive cardiomyocytes (6- and 2-fold respectively) as compared to AAV-injected hearts. Notably, non-cardiomyocytes were rarely positive for mCherry, which is consistent with published study of AAV9 selectively expression in cardiomyocytes. In a parallel experiment examined AAVExo and AAV transduction were examined through tail vein administration. Similarly improved expression of AAVExo was confirmed in both left ventricle and right ventricle.

Intramyocardially or intravenously injected AAV9Exo-mCherry or AV9-mCherry (3-4E10 viral genomes) demonstrates significantly higher expression in CMs compared to non-CMs after 2 weeks. Moreover, AAVExo-injected hearts had significantly higher mCherry-positive CMs compared to free AAV-injected hearts (FIGS. 4E-4G) (the low % of mCherry+ve cells were due to low starting virus titer). Interestingly, similar trends were not observed in non-CMs. Together, these data indicate robust enhancement of gene transfer to CMs in vivo with AAVExo vectors compared with free AAV. These data also suggest that AAVExo preserve cardiotropic properties of AAV9 serotype57.

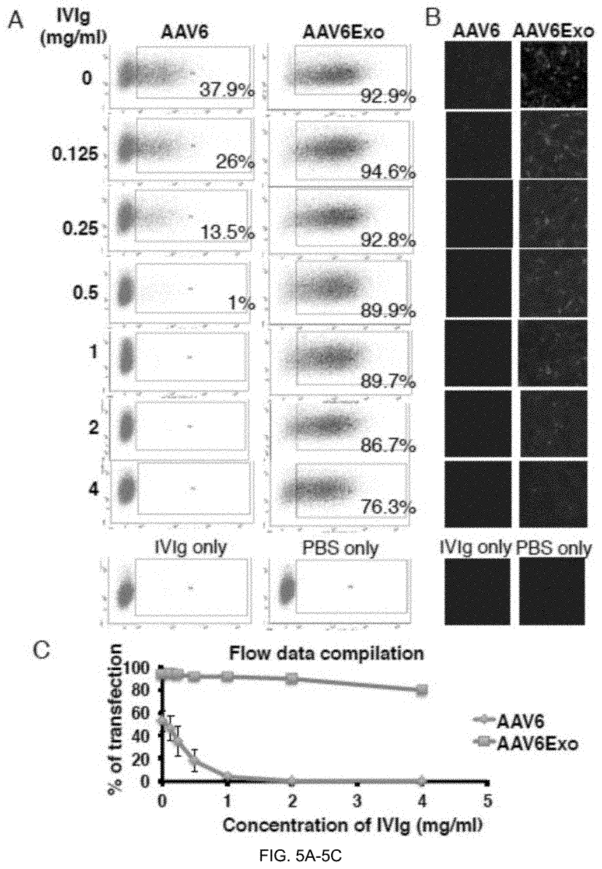

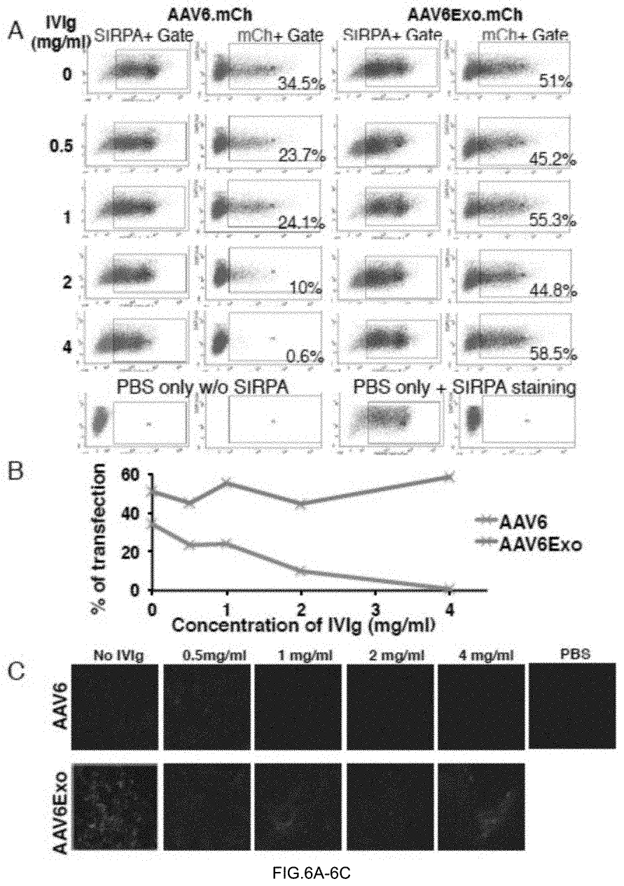

AAVExo is more resistant to antibody neutralization compared to AAV in cultured human cardiomyocytes in vitro: Pre-existence of neutralizing antibody (Nab) against AAV is prevalent in human serum. Nab binds to AAV, blocks its infection and impairs AAV-mediated gene delivery. To determine whether AAVExo is resistant to Nab neutralization, equal titer of AAV6Exo-mCherry or AAV6-mCherry was pre-incubated with dilutions of Nab (human intravenous immunoglobulin (IVIg)) or PBS control at 37° C. for 30 min before applied on AC16 human ventricular cardiomyocytes. AAV6 was used for the reason that it has better transduction rate than AAV9 for in vitro system. After 3 days, mCherry expression was examined by flow cytometry and confocal microscopy (FIGS. 5A, 5B). Percentage of transduced cells was plotted from flow cytometry (FIG. 5C). As expected, significant reduction of mCherry expression was observed for Nab-incubated AAV (˜0% at 1 mg/ml IVIg). However, there was no significant decrease in AAVExo-mCherry expression in presence of Nab (>75%). To confirm these data, a similar experiment was on human iPS-derived cardiomyocytes (iPS-CM). After transduction, iPS-CM were stained with SIRPA, a surface protein marker for induced CM37, and analyzed by flow cytometry (FIGS. 6A, 6B) and confocal microscopy (FIG. 6C). Remarkably, AAVExo retained drastically high transduction efficiency at high concentration of Nab (58% at 4 mg/ml IVIg), whereas AAV transduction was completely blocked. These data provide strong evidence that AAVExo but not AAVs can resist neutralization by Nab.

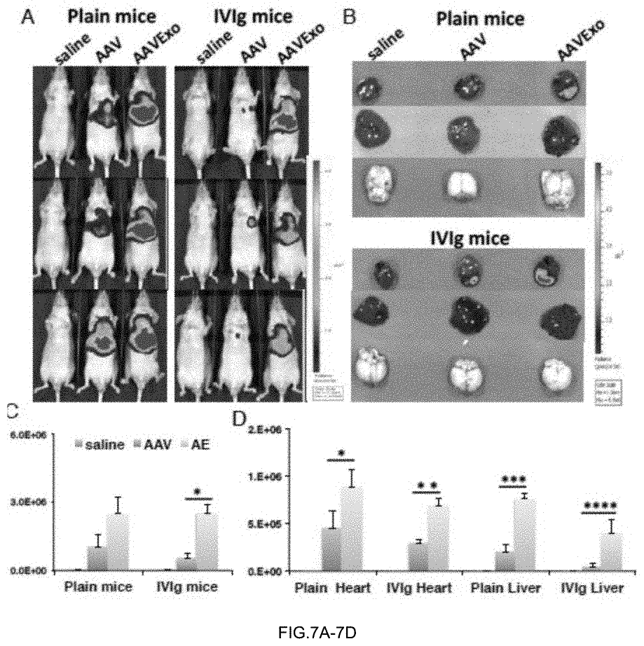

AAVExo is more resistant to antibody neutralization compared to AAV in vivo: Next the resistance of AAVExo to Nab neutralization was tested in a rodent model. To generate animal model with pre-existing Nab, nude mice (nu/J) were i.p injected with IVIg (1 mg/mouse) or PBS (plain mice). Neutralizing effect of the serum from IVIg-injected mice through in vitro assay was further confirmed. After 24 hours, IVIg mice or plain mice were intramyocardially injected with AAV9Exo-FLuc, AAV9-FLuc (5E¹⁰ g.c. per mouse, saline as a control). One, two and four weeks later live mice were imaged for firefly luciferase (FLuc) expression by bioluminescent imaging (FIG. 7A). Intensity of bioluminescence from the area of chest/heart was quantified (FIG. 7C). Consistent to in vitro data, AAVExo presented the gene-delivery efficiency above two-fold higher than AAV in plain mice. In mice pre-injected with IVIg, AAV transduction was largely inhibited as expected, while, remarkably, in IVIg mice AAVExo presented similar level of gene-transfer efficiency as in plain mice (FIG. 7C). In addition to live imaging, mouse heart and other organs including liver, spleen, brain, kidney, and lung were collected after 4 weeks and imaged ex vivo for FLuc expression (heart, liver and brain). Intensity of bioluminescence in heart and liver was quantified in FIG. 7B, D. In line with in vivo data, AAVExo remained significantly higher level of transduction efficiency in mice with IVIg as opposed to the reduction of AAV. The trend of FLuc expression in liver was consistent to heart, validating the robust resistance of AAVExo to Nab. In other organs, including brain, FLuc expressions were not detected. These data strongly demonstrated enhanced gene-delivery efficiency of AAVExo than conventional AAV in rodent model. In addition, gene delivery was minimal in other organs tested (liver, lung and spleen, data not shown), suggesting that majority of injected vectors were retained in myocardium. Together, these results demonstrate that AAVExo had significantly higher transduction efficiency compared to AAV both in vitro and in vivo. Remarkably in presence of Nab, as opposed to AAV, AAVExo still maintain comparable level of gene transfer in vivo instead of being significantly blocked by Nab.

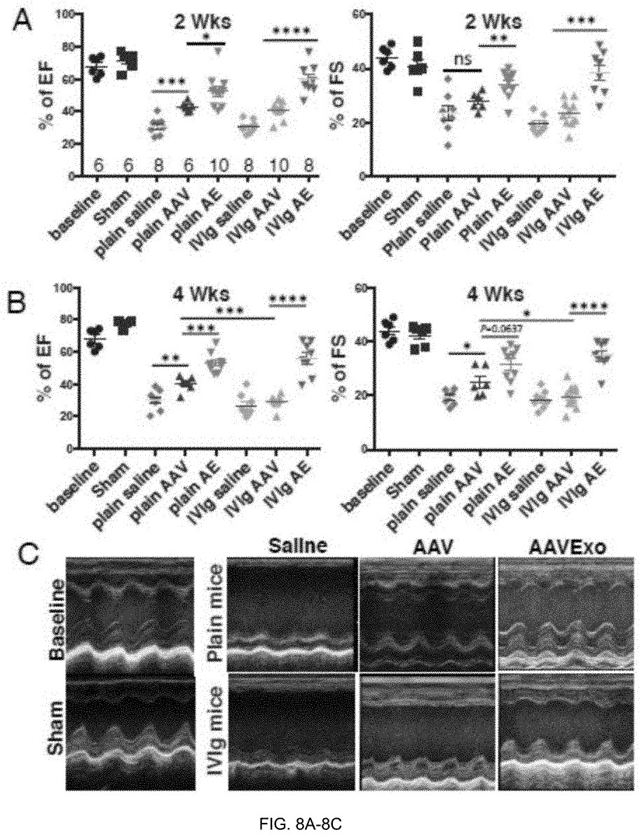

AAVExo-SERCA2a significantly improved cardiac function in post-MI hearts even in presence of neutralizing antibody: Next Improved therapeutic benefits of AAVExo as to AAV in a preclinical mouse model with myocardial infarction (MI) in presence or absence of preexisting Nab was next evaluated. SERCA2a plays a major role in regulating calcium level in cardiomyocytes. Reduction in SERCA2a activity closely associates with contractile dysfunction and heart failure. SERCA2a gene delivery using AAV has been shown to reverse contractile dysfunction and myocardial remodeling in rodent and large animal models. However, AAV neutralization by the presence of neutralizing antibody is a persistent challenge to maximize the clinical benefits of SERCA2a gene transfer to treat patients with heart failure. Herein AAV9Exo-SERCA2a and AAV9-SERCA2a were generated as a control using previously published methods. Nude mice were i.v. injected with IVIg or same volume of saline 24 hours before MI surgery, for which left anterior descending artery was ligated to form an ischemia. Mice with same procedure but no ligation were used as sham control. Immediately after surgery, same titer of AAV9Exo-SERCA2a (1E¹¹ g.c.), AAV9-SERCA2a or saline was directly injected to left ventricle tissue around ischemic zone of MI mice. Heart ejection fraction (EF) and fractional shortening (FS) were evaluated by echocardiography at 2, 4 and 6 weeks post-surgery (FIGS. 8A, 8B). In plain mice, which didn't have Nab, AAV9-SERCA2a could improve cardiac functions to some extent, whereas heart enhancement by AAV9Exo was significantly higher compared to AAV9. In IVIg mice, EF and FS of AAV-administered mice were significantly lower than those of plain mice, due to expected IVIg neutralization to AAV (FIGS. 8A, 8B). However, beneficial effects of AAVExo in IVIg mice retained at similar level as in plain mice, and, obviously, significantly greater than AAV in IVIg mice. Consistent results were seen in left ventricle wall thickness and movement (FIG. 8C). Overall, the in vivo functional data reveals that AAVExo presented better effects than AAV in protecting heart functions in mice with MI, and more importantly, AAVExo was resistant to Nab and remained protective potential for cardiac functions in Nab-existing mice.

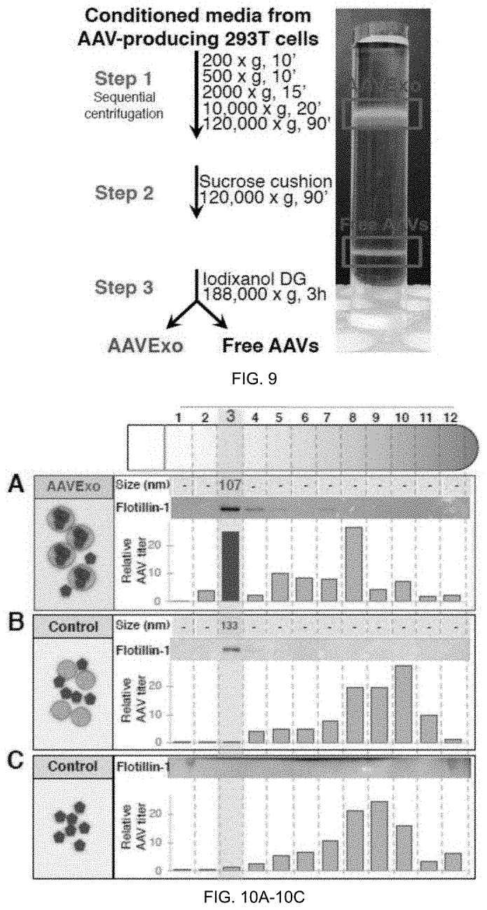

An alternative embodiment of a method shown in FIG. 1A is shown in FIG. 9. In this non-limiting example, sequential centrigugation steps to separate AAVExo from cellular debris were performed as follows: 10 min at 200 g, 10 min at 500 g, 15 min at 2,000 g, 20 min at 10,000 g, and 90 min at 120,000 g, in accordance with aspects of the present disclosure; centrifugation on sucrose cushion, in accordance with aspects of the present disclosure, was for 90 min at 120,000 g; and centrifugation on an iodixanol gradient, in accordance with aspects of the present disclosure, was for 3 h at 188,000 g.

Purified AAVExo fraction was shown to relatively be free AAV as shown by DLS size measurements. Again, three different conditions were in parallel and loaded on to density gradient step: 1) AAVExo-containing conditioned media (FIG. 10A), 2) free AAVs (purified from the cells, FIG. 10C), and 3) a mixture of free AAVs and empty exosomes (from untransfected HEK cells) (FIG. 10B). The total genome copy number (GCN) of AAVs and the amount of exosomes (determined by protein content) loaded were equal in all conditions. After ultracentrifugation, twelve 1 ml fractions were collected from the top of each density gradient, and analyzed for the presence of exosomes (DLS for size and WB for exosome marker protein-flotillin-1) and for the AAV GCN (by qPCR; FIGS. 10A-10C). In this preparation, DLS and WB data indicated that exosomes were primarily located in post-centrifugation fraction 3 (F3, in red). Interestingly, significant quantity of AAV genomes was detected in F3 in the conditioned media containing AAVExo (FIG. 10A), but not in the F3 of two other control samples. This suggests that purification was successful in minimizing the presence of free AAV in an isolated AAVExo fraction.

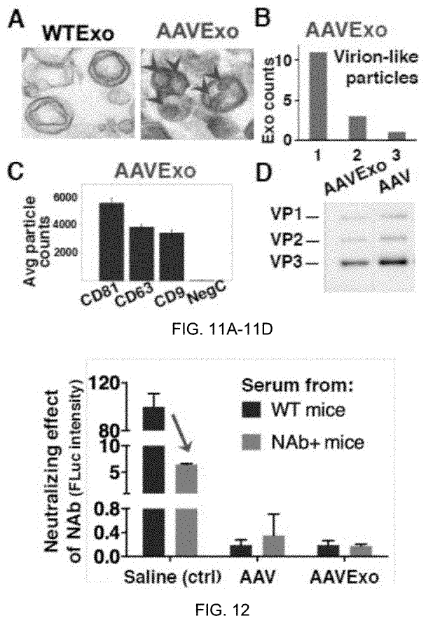

Characterization of AAVExo: DLS analysis of AAVExo contained in F3 showed an average size of ˜100 nm (FIGS. 1C and 10A). FIG. 11A shows representative TEM micrographs of ultrathin sections of exosome pellets of WTExo (left) and AAVExo (right; red arrows indicate AAVs inside exosomes). FIG. 11B shows distribution of number of virion-like particles contained within individual AAVExo. FIG. 11C shows concentration of AAVExo immobilized using different antibodies on the ExoView Tetraspanin Assay Chip (NanoView Biosciences). FIG. 11D shows western blot analysis for the AAV capsid proteins VP1, VP2, and VP3. ExoView Platform demonstrated the presence of known exosomes surface markers such as CD81, CD63 and CD9 (FIG. 11B), and Western blot analysis confirmed the presence of AAV capsid proteins (VP1, VP2 and VP3; FIG. 11D) and exosomes marker, flotilin (FIG. 10A). Interestingly, TEM analysis of the ultrathin sections of exosomes pellet demonstrates presence of viral capsids inside the lumen of AAVExo, but not inside the WTexo (FIGS. 11A-11B). Taken together, our preliminary results suggest that AAVExo purified in post-centrifugation F3 are relatively pure with no significant contamination from free AAV vectors.

Passive immunity nude mouse model efficiently mimic naturally-occuring immunity to AAV and its effects on AAV-mediated gene transfer: To further investigate AAVExo transduction efficiency in vivo, an animal model with pre-existing AAV immunity was generated by intraperitoneally injecting human intravenous immunoglobulins (1 mg/mouse; Bax) to nude mice, and compared the neutralizing effect of NAb present in the serum of NAb pre-injected mice and WT (c57b1) mice. Interestingly, serum isolated from NAb pre-injected mice inhibited AAV9 in vitro transduction compared to serum derived from non-infected WT mice, indicating the utility of this experimental model in our further AAV studies (FIG. 12). Moreover, sera from AAV- and AAVExo-injected WT mice completely inhibited AAV trandsuction in vitro, suggesting that administration of AAV or AAVExo enhanced immunity to AAV in both mice species (FIG. 12). Neutralizing effect of the serum from NAb pre-injected mice was therefore confirmed.

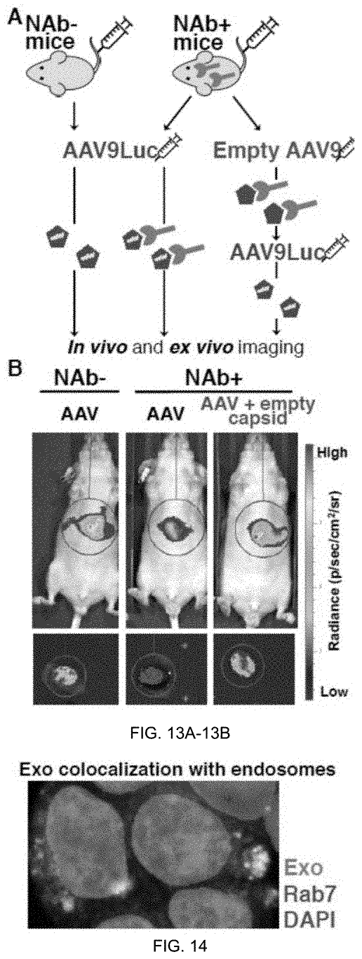

Empty virus capsids overcome NAb-mediated immunity to AAV: Empty virus capsids compete with AAV virus particles for Nab binding, and thereby and thereby can increase AAV transduction in presence of NAb. Passivei immunization occurred with NAb injected intraperitoneally 24 hours before administration of AAV9-Luc or empty AAV9 capsids (3E11) followed by AAV9-Luc (FIG. 13A). NAb+ mice were imaged for firefly luciferase (FLuc) expression by whole animal bioluminescent imaging (BLI) using IVIS Platform and compared the intensity of bioluminescence from the chest area of these animals with NAb− mice injected with AAV9-Luc. In mice pre-injected with NAbs, AAV-mediated gene delivery was largely inhibited. Remarkably, mice simultaneously injected with same titer of empty capsids and virus vectors showed similar transduction efficiency as NAb− mice (FIG. 13B). Results from ex vivo heart imaging demonstrated similar results, suggesting that AAV empty capsids allow for efficient vector delivery in the presence of NAb. Thus, empty capsids serve as highly effective decoys for antibodies to AAV (Nab), and overcoming preexisting humoral immunity.

In FIG. 14, shown is cellular trafficking of HEK293 cell-derived exosomes. Confocal microscopy analysis showed colocalization of fluorescent dye-labeled exosomes and endosomes (immunolabeled with Rab7 antibody) in HEK293T cells.

Although preferred embodiments have been depicted and described in detail herein, it will be apparent to those skilled in the relevant art that various modifications, additions, substitutions, and the like can be made without departing from the spirit of the present disclosure and these are therefore considered to be within the scope of the present disclosure as defined in the claims that follow.

What is claimed is:

1. A method of making isolated exosomes containing an adeno-associated viral (AAV) vector, comprising
disposing a suspension on an iodixanol gradient, wherein the suspension comprises exosomes containing AAV vectors and the iodixanol gradient comprises a higher layer of between 20% and 30% w/v iodixanol solution, an intermediate layer of between 30% and 50% w/v iodixanol solution, and a lower layer of between 50% and 70% w/v iodixanol solution, and
centrifuging the iodixanol gradient at between 180,000 g and 300,000 g for at least 2 hours.

2. The method of claim 1, further comprising, before disposing the suspension on the iodixanol gradient, forming a sucrose suspension by suspending exosomes containing AAV vectors in a 30% w/v solution of sucrose in deuterium oxide and centrifuging the first suspension at between 80,000 g and 120,000 g for at least 70 min to form a fraction of exosomes containing AAV vectors, washing the fraction, and suspending the fraction in a solution that does not contain sucrose.

3. The method of claim 1 or claim 2 wherein cells in which the exosomes containing AAV vectors were produced comprise 293T cells, Per.C6 cells, AGE1.CR cells, AGE1.HN cells, KG-1 cells, human umbilical vein endothelial cells (HUVECs), embryonic stem (ES) cells from humans, human mesenchymal stem cells (MSCs), human fibroblasts, human keratinocytes, human hematopoietic progenitor cells, human cKit+ stem cells, human cardiosphere-derived cells, HeLa cells, induced pluripotent cells, K562 cells, Caco-2 cells, human T-cells, human dendritic cells, human smooth muscle cells, or endothelial cells derived from vascular origin.

4. The method of claim 1 wherein the lower layer comprises 60% w/v iodixanol solution, the intermediate layer comprises 40% w/v iodixanol solution, and the higher layer comprises 25% w/v iodixanol solution.

5. The method of claim 1 comprising centrifuging the iodixanol gradient at 250,000 g.

6. The method of claim 1 comprising centrifuging the iodixanol gradient for at least 3 hours.

7. The method of any of claim 1, 2, 4, 5, or 6, further comprising collecting a final fraction from the iodixanol gradient after centrifuging the iodixanol gradient, wherein the final fraction comprises a highest portion of the first layer and the portion is up to one half volume of the higher layer.

8. The method of claim 7, wherein the portion is up to one quarter volume of the higher layer.

9. The method of claim 7, wherein the portion is up to one eighth volume of the higher layer.

10. The method of claim 7 wherein more than 95% of AAV vector genome copies present in the final fraction are in exosomes.

11. The method of claim 8 wherein more than 95% of AAV vector genome copies present in the final fraction are in exosomes.

12. The method of claim 9 wherein more than 95% of AAV vector genome copies present in the final fraction are in exosomes.

13. The method of any one of claim 1, 2, 4, 5, or 6, wherein the AAV vectors are AAV1, AAV2, AAV6, or AAV9 vectors.

14. The method of claim 8, wherein the AAV vectors are AAV1, AAV2, AAV6, or AAV9 vectors.

15. The method of claim 9, wherein the AAV vectors are AAV1, AAV2, AAV6, or AAV9 vectors.

16. A method of transfecting a cell, comprising contacting the cell with exosomes containing an adeno-associated viral (AAV) vector, wherein a method for making the exosomes containing an AAV vector comprises:
disposing a suspension on an iodixanol gradient, wherein the suspension comprises exosomes containing AAV vectors and the iodixanol gradient comprises a higher layer of between 20% and 30% w/v iodixanol solution, an intermediate layer of between 30% and 50% w/v iodixanol solution, and a lower layer of between 50% and 70% w/v iodixanol solution
centrifuging the iodixanol gradient at between 180,000 g and 300,000 g for at least 2 hours.

17. The method of claim 16, further comprising, before disposing the suspension on the iodixanol gradient, forming a sucrose suspension by suspending exosomes containing AAV vectors in a 30% w/v solution of sucrose in deuterium oxide and centrifuging the first suspension at between 80,000 g and 120,000 g for at least 70 min to form a fraction of exosomes containing AAV vectors, washing the fraction, and suspending the fraction in a solution that does not contain sucrose.

18. The method of claim 16 or claim 17 wherein cells in which the exosomes containing AAV vectors were produced comprise 293T cells, Per.C6 cells, AGE1.CR cells, AGE1.HN cells, KG-1 cells, human umbilical vein endothelial cells (HUVECs), embryonic stem (ES) cells from humans, human mesenchymal stem cells (MSCs), human fibroblasts, human keratinocytes, human hematopoietic progenitor cells, human cKit+ stem cells, human cardiosphere-derived cells, HeLa cells, induced pluripotent cells, K562 cells, Caco-2 cells, human T-cells, human dendritic cells, human smooth muscle cells, or endothelial cells derived from vascular origin.

19. The method of claim 16 wherein the lower layer comprises 60% w/v iodixanol solution, the intermediate layer comprises 40% w/v iodixanol solution, and the higher layer comprises 25% w/v iodixanol solution.

20. The method of claim 16 comprising centrifuging the iodixanol gradient at 250,000 g.

21. The method of claim 16 comprising centrifuging the iodixanol gradient for at least 3 hours.

22. The method of any of claim 16, 17, 19, 20, or 21, further comprising collecting a final fraction from the iodixanol gradient after centrifuging the iodixanol gradient, wherein the final fraction comprises a highest portion of the higher layer and the portion is up to one half volume of the higher layer.

23. The method of claim 22, wherein the portion is up to one quarter volume of the higher layer.

24. The method of claim 22, wherein the portion is up to one eighth volume of the higher layer.

25. The method of claim 22 wherein more than 95% of AAV vector genome copies present in the final fraction are in exosomes.

26. The method of claim 23 wherein more than 95% of AAV vector genome copies present in the final fraction are in exosomes.

27. The method of claim 24 wherein more than 95% of AAV vector genome copies present in the final fraction are in exosomes.

28. The method of any one of claim 16, 17, 19, 20, or 21, wherein the AAV vectors are AAV1, AAV2, AAV6, or AAV9 vectors.

29. The method of any one of claim 16, 17, 19, 20, or 21, wherein the AAV vectors are AAV9 vectors.

30. A method of making isolated exosomes containing an adeno-associated viral (AAV) vector, comprising
forming a sucrose suspension by suspending exosomes containing AAV vectors in a 30% w/v solution of sucrose in deuterium oxide and centrifuging the first suspension at 100,000 g for at least 70 min to form a fraction of exosomes containing AAV vectors, washing the fraction, and suspending the fraction in a solution that does not contain sucrose, forming a suspension,
disposing a suspension on an iodixanol gradient, wherein the suspension comprises exosomes containing AAV vectors and the iodixanol gradient comprises a higher layer of 25% w/v iodixanol solution, an intermediate layer of 40% w/v iodixanol solution, and a lower layer of 60% w/v iodixanol solution,
centrifuging the iodixanol gradient at 250,000 g for at least 3 hours, and
collecting a final fraction from the iodixanol gradient after centrifuging the iodixanol gradient, wherein the final fraction comprises a highest portion of the first layer and the portion is up to one eighth volume of the higher layer,
wherein the AAV vectors are AAV9 vectors and cells in which the exosomes containing AAV vectors were produced comprise 293T cells.

31. A suspension comprising exosomes containing adeno-associated viral (AAV) vectors, wherein 90% or more of AAV vector genome copies present in the suspension are in exosomes.

32. The suspension of claim 31, wherein 95% or more of AAV vector genome copies present in the suspension are in exosomes.

33. The suspension of claim 31, wherein 97.5% or more of AAV vector genome copies present in the suspension are in exosomes.

34. The suspension of claim 31, wherein the AAV vectors are AAV1, AAV2, AAV6, or AAV9 vectors.

35. The method of claim 31 wherein the AAV vectors are AAV9 vectors.
